(19) United States
(12) Patent Application Publication
(10) Pub. No.: US 2019/0070229 A1
(43) Pub. Date: Mar. 7, 2019

(54) Composition comprising a lactic acid bacteria for preventing and treating vaginosis and the use thereof

(71) Applicant: HAUDONGCHUN CO., LTD, Seoul (KR)

(72) Inventors: Won Seog CHOI, Seoul (KR); Moo Hyung LEE, Seoul (KR)

(21) Appl. No.: 16/068,871

(22) PCT Filed: Apr. 26, 2017
(86) PCT No.: PCT/KR2017/004408

§ 371 Date: Jul. 9, 2018

(30) Foreign Application Priority Data
May 10, 2016 (KR) 10-2016-0057017
Sep. 8, 2016 (KR) 10-2016-0115716

(51) Int. Cl. A61K 35/747; A61K 35/745; A61K 9/00; A61K 47/02; A61K 47/26; A61K 47/38; A61P 15/02; A23L 33/135

(52) U.S. Cl. CPC A61K 35/747; A23Y 2300/55; A61K 9/0034; A61K 47/02; A61K 47/26; A61K 47/38; A61P 15/02; A23L 33/135; A23Y 2220/03; A23Y 2220/17; A23Y 2220/63; A23Y 2220/67; A23Y 2220/65; A23Y 2220/73; A23Y 2300/19; A23Y 2300/29; A23Y 2300/59; A23Y 2300/65; A23Y 2300/25; A23Y 2300/45; A23Y 2300/31; A23Y 2300/49; A61K 35/745

(57) ABSTRACT

A composition comprising combination of salt, sugar and lactic acid bacteria as an active ingredient to treat vaginosis. The composition showed potent antibacterial activity through various experiments, for example, (1) the indirect inhibitory activity test from the growth of vaginosis causing bacteria by determining the change of pH and lactic acid level; (2) the direct inhibitory activity test from the growth of vaginosis causing bacteria by determining the susceptibility of test sample; (3) brief clinical tests, and finally confirmed that the combination showed potent antibacterial activity in the test. Accordingly, the combination may be useful to alleviate and treat vaginosis in the form of a pharmaceutical composition, health functional food, food additive, topical composition, and detergent composition.

### TECHNICAL FIELD

The present invention relates to a composition comprising a salt, sugar and lactic acid bacteria as active ingredients for preventing and treating vaginosis and the use thereof.

### BACKGROUND ART

Vaginitis is a condition that occurs especially during pregnancy in the vagina causing vaginal discharge, inflammation, and irritation, as well as vulvar or vaginal itching. The three most common vaginal infections and diseases are also the most frequent causes of vaginitis. The three common vaginal infections include: bacterial vaginosis, vaginal yeast infection, and trichomoniasis.

The human vagina is colonized with various microbes, yeast and germ, for example, about more than 10⁴ numbers/ml (vaginal fluid) of Lactobacillus spp such as Lactobacillus crispatus and Lactobacillus jensenii, which provide weak acidic environment ranging from pH 4.5-5.1 to protect from microbial infection and is a highly versatile organ that can profoundly affect the health of women and their newborn infants. There have been reported that there are many important pathogens in the vaginal niche such as Neiserria gonorrhea, Ureaplasma species, Mycoplasma genitalium, Streptococcus species, Escherichia coli, Chlamydia trachomatis, and Trichomonas vaginalis etc.

Especially, bacterial vaginosis (BV), the most prevalent and detrimental vaginosis, gives rise to malodorous vaginal discharge or local irritation, of the women with BV and is associated with several more serious adverse outcomes including preterm birth, pelvic inflammatory disease, and acquisition of HIV infection. The women with the condition bacterial vaginosis (BV) have loss of many Lactobacillus species (except L. iners) and acquisition of a variety of anaerobic and facultative bacteria. Gram stains of vaginal fluid from women with BV show loss of Gram-positive rods and their replacement with Gram-negative and Gram-variable cocci and rods. Cultures of vaginal fluid from subjects with BV typically yield Gardnerella vaginalis and a mixture of other bacteria that may include Peptosterptococcus, Mobiluncus, Bacterioides, Prevotella, Porphyromonas, Mobiluncus and Mycoplasma species. (Sujatha Srinivasan and David N. Fedricks, Review Article, The Human Vaginal Bacterial Biota and Bacterial Vaginosis, Interdisciplinary Perspectives on Infectious Diseases, Vol., 2008, Article ID 750479, p 1-3).

Lactic acid bacteria, frequently found in the intestine of human or animal, fermented food etc, has been reported as a representative bacteria useful as a probiotic agent and as being acknowledged as a safe bacteria by FDA (Food and Drug Administration) (Orrhge, K. et al., 2000, Bifidobateria and lactobacilli in human health, Drug Experimental Clinical Research., 26, pp 95-111). Lactic acid bacteria being adhered to intestinal epithelial cell and being parasitic, improves the environment of gut microbiota and provides host animals with various beneficial advantages, for example, stabilization of gut microbiota, decrease of decomposing matter by dint of inhibiting from the adherence of intestinal harmful bacteria, prevention of harmful disease, immune-activating activity, anti-cancer activity, cholesterol lowering activity, etc. The growth inhibiting activity of lactic acid bacteria from various spoilage microorganism and pathogenic microorganism is reported to be due to the metabolic characteristics of their metabolites releasing antibacterial factors, for example, organic acid, hydrogen peroxide, reuterin, diacetyl, acetaldehyde, bacteriocin and the like (Fuller, K., 1989, Probiotics in man and animals, J. Appl. Bacteriol., 66, pp 365-378; Korean Patent Publication No. 10-2015-0075447 A).

There have been studied to develop effective therapies to treat vaginitis, for example, orally administrated broad spectrum antibiotics such as metronidazole till now. However the therapy shows lots of disadvantages such as antibiotics intolerance, systemic toxicity in case of long-term administration, and a probable destruction of normal bacterial flora in vagina causing to secondary complication such as a decreased number of lactobacillus spp., an increase of vaginal pH, and a proliferation of anaerobic microbes etc.

Accordingly, there has been needed to develop novel therapeutic composition showing long-term treating activity with safety and little adverse response to treat vaginosis till now. The present inventors had found that the composition comprising combinations of salt and sugar as active ingredients showed potent anti-bacterial activity against and proliferating activity (Korea patent Registration No. 10-1133723 B1/PCT/WO2011/049327 A1); and potent treating effect on lax vagina syndrome or colpoxerosis disease (Korea patent Registration No. 10-1470282 B1/PCT/WO 2015/050324 A1) till now.

However, there has been not reported or disclosed on the therapeutic effect for vaginosis of the combination of a salt, sugar and lactic acid bacteria in any of the above cited literatures, the disclosures of which are incorporated herein by reference.

To investigate an inhibitory effect of the combination of salt, sugar and lactic acid bacteria on vaginosis, the inventors of the present invention have carried out (1) the indirect inhibitory activity test from the growth of vaginosis causing bacteria by determining the change of pH and lactic acid level (Experimental example 1); (2) the direct inhibitory activity test from the growth of vaginosis causing bacteria by determining the susceptibility of test sample (Experimental example 2); (3) brief clinical tests, and finally completed present invention by confirming that the combination showed potent antibacterial activity in the test.

These and other objects of the present invention will become apparent from the detailed disclosure of the present invention provided hereinafter.

### DISCLOSURE

### Technical Problem

Accordingly, it is another object of the present invention to provide a pharmaceutical composition comprising a combination of salt, sugar and lactic acid bacteria as an active ingredient to treat or prevent vaginosis.

It is another object of the present invention to provide a use of a combination of salt, sugar and lactic acid bacteria in the manufacture of a medicament employed for treating or preventing vaginosis in a mammal.

It is the other object of the present invention to provide a method of treating or preventing vaginosis in a mammal wherein method comprises administering to said mammal an effective amount of a combination of salt, sugar and lactic acid bacteria, together with a pharmaceutically acceptable carrier thereof.

It is the other object of the present invention provides a health functional food comprising a combination of salt, sugar and lactic acid bacteria as an active ingredient to alleviate or prevent vaginosis.

It is the other object of the present invention provides a health care food comprising a combination of salt, sugar and lactic acid bacteria as an active ingredient to alleviate or prevent vaginosis.

It is the other object of the present invention provides a food additive comprising a combination of salt, sugar and lactic acid bacteria as an active ingredient to alleviate or prevent vaginosis.

It is the other object of the present invention provides a topical composition comprising a combination of salt, sugar and lactic acid bacteria as an active ingredient to treat or prevent vaginosis.

It is the other object of the present invention provides a detergent composition comprising a combination of salt, sugar and lactic acid bacteria as an active ingredient to alleviate or prevent vaginosis.

### Technical Solution

In one embodiment of the present invention, the present invention provides a pharmaceutical composition comprising a combination of salt, sugar and lactic acid bacteria as an active ingredient for the treatment and prevention of vaginosis.

The present invention provides a pharmaceutical composition comprising a combination of salt, sugar and lactic acid bacteria as an active ingredient for the treatment and prevention of vaginosis, together with a pharmaceutically acceptable carrier or excipients.

Additionally, the present invention provides a use of a combination of salt, sugar and lactic acid bacteria in the manufacture of a medicament employed for treating or preventing vaginosis in a mammal.

Additionally, the present invention provides a method of treating or preventing vaginosis in a mammal wherein method comprises administering to said mammal an effective amount of a combination of salt, sugar and lactic acid bacteria together with a pharmaceutically acceptable carrier thereof.

The term, “salt” defined herein comprise a refined salt, processed salt such as melted salt, or an un-refined salt such as sea salt, rock-salt etc; preferably, a refined salt such as sodium chloride or melted salt such as bamboo salt, more preferably, a melted salt prepared by melting a un-refined salt at the temperature ranging from 200 to 2000° C., preferably, from 800 to 1200° C., for the period ranging from 2 hours to 7 days, preferably, 12 hours to 48 hours.

The term, “sugar” defined herein comprise a saccharide compound known to be as a useful prebiotics in the art, preferably, mono-saccharides, disaccharides, oligosaccharide, polysaccharide etc, more preferably, mono-saccharides comprising a pentose such as xylose, arabinose and the like; hexose such as glucose, mannose, fructose, galactose and the like; disaccharides such as lactulose, lactitol, sucrose, lactose, maltose, trehalose and the like; oligosaccharides such as fructo-oligosaccharide, raffinose, stachyose, maltodextrin and the like; polysaccharide such as amylose, cellulose, pectin and the like, more preferably, a saccharide selected from glucose, fructose, galatose, lactulose, lactitol, sucrose, lactose, or fructo-oligosaccharide.

The term, “lactic acid bacteria” defined herein comprise any generally known or available lactic acid as probiotics in the art, preferably, any conventionally available lactic acid as probiotics from the internationally approved depository authorities such as International depository authority (IDA), for example, KCTC (Korean Collection for Type Cultures), KCCM (Korean Culture Center of Microorganisms) or KACC (Korean Agricultural Culture Collection) in South Korea; NRRL (Agricultural Research Service Culture Collection) or ATCC (American Type Culture Collection), NCMA (Provasoli-Guillard National Center for Marine Algae and Microbiota) in U.S.A; CCAP (Culture Collection of Algae and protozoa), ECACC (European Collection of Cell Cultures), IMI (International Mycological Institute), NCTC (National Collection of Type Culture), NCYC (National Collection of Yeast Cultures), NCIMB (National Collections of Industria, Food and Marine Bacteria), or NIBSC (National Institute for Biological Standards and Controls) in Great Britain; CNCM (Collection Nationale De Cultures de Microorganisms) in France; DSMZ (Lebniz-Institu DSMZ-Deutsche Sammlung von Mikroorganismen and Zellkulturen GmbH) in Germany; IPOD (International patent organism Depository), NPMD (National Institute of Technology and Evaluation, Patent Microorganism Depository) in Japan; CCTCC (China Center for Culture Collection), CGMCC (China General Microbiological Culture Collection Center) in China etc; specifically, any lactic acid bacteria belonged to Lactobacillus spp., Bifidobacterium spp., Bacillus spp., Streptococcus spp., Enterococcus spp. Saccharomyces spp., Leuconostoc spp. etc, more specifically, any lactic acid bacteria selected from the group consisting of Lactobacillus spp., such as lactobacillus plantarum, lactobacillus pentosus, lactobacillus casei, lactobacillus casei ssp. paracasei, lactobacillus rhamnosus, lactobacillus acidophilus, lactobacillus delbrueckii, lactobacillus delbrueckii, ssp. bulgaricus, lactobacillus delbrueckii, ssp. delbrueckii, lactobacillus fermentum, lactobacillus gasseri, lactobacillus reuteri, lactobacillus brevis, lactobacillus cellobiosus, lactobacillus crispatusi, lactobacillus GG, lactobacillus johnsonii, lactobacillus lactis, lactobacillus salivarius and the like; Bifidobacterium spp. such as Bifidobacterium longum, Bifidobacterium bifidum, Bifidobacterium bereve, Bifidobacterium animalis ssp, lactis, Bifidobacterium adoescentis, Bifidobacterium pseuocatenulatum, Bifidobacterium catenulatum, Bifidobacterium infantis, Bifidobacterium thermophilum and the like; Bacillus spp., such as Bacillus cereus toyoi, Bacillus cereus and the like; Streptococcus spp., such as Streptococcus thermophiles, Streptococcus cremoris, Streptococcus infantarius, Streptococcus intermedius, Streptococcus lactis, Streptococcus salivarius subsp. Thermophiles, and the like; Enterococcus spp. such as Enterococcus faecalis, Enterococcus faecium, and the like; Saccharomyces spp., such as Saccharomyces cerevisiae, Saccharomyces boulardii and the like; Leuconostoc spp such as Leuconostoc citreum, Leuconostoc mesenteroides and the like; more specifically, Lactobacillus spp., such as lactobacillus plantarum, lactobacillus pentosus, lactobacillus casei, lactobacillus casei ssp. paracasei, lactobacillus rhamnosus, or lactobacillus acidophilus; Bifidobacterium spp. such as Bifidobacterium longum, Bifidobacterium bifidum, or Bifidobacterium bereve; Bacillus spp., such as Bacillus cereus toyoi, or Bacillus cereus; Streptococcus spp., such as Streptococcus thermophiles, Streptococcus cremoris, Streptococcus infantarius, Streptococcus intermedius, or Streptococcus lactis.

The term, “a combination of salt, sugar and lactic acid bacteria” defined herein comprise a combination of salt, sugar and lactic acid bacteria mixed ratio of 1:100-0.01:100-0.01 weight part (w/w), preferably, 1:50-0.5:50-0.5 weight part (w/w), more preferably, 1:30-0.3:30-0.3 weight part (w/w), more and more preferably, 1:10-0.1:10-0.1 weight part (w/w), most preferably, 1:5-1:5-1 weight part (w/w).

The composition of the present invention may further contain the other antibiotics, dye, flavor etc in the amount of about 0.1˜20% by weight of the above composition based on the total weight of the composition.

Hereinafter, the present invention is described in detail.

An inventive composition comprising the combination of salt, sugar and lactic acid bacteria can be prepared in detail by following procedures,

For example, the inventive cleansing combination of the present invention can be prepared by follows; preparing a refined salt, processed salt such as melted salt, or an un-refined salt such as sea salt, rock-salt etc, preferably, refined salt or processed salt prepared by melting un-refined salt at the temperature ranging from 200 to 2000° C., preferably, from 800 to 1200° C., for the period ranging from 2 hours to 7 days, preferably, 12 hours to 48 hours to obtain the melted salt at the 1^{st} step; the melted salt is mixed with sugar compound such as mono-saccharides, disaccharides, oligosaccharide, polysaccharide etc, and lactic acid bacteria belonged to Lactobacillus spp., Bifidobacterium spp., Bacillus spp., Streptococcus spp., Enterococcus spp. Saccharomyces spp., Leuconostoc spp. etc, with mixed ratio of mixed ratio of 1:100-0.01:100-0.01 weight part (w/w), preferably, 1: 50-0.5:50-0.5 weight part (w/w), more preferably, 1:30-0.3:30-0.3 weight part (w/w), more and more preferably, 1:10-0.1:10-0.1 weight part (w/w), most preferably, 1:5-1:5-1 weight part (w/w) to obtain inventive combination; and the combination is dissolved in an appropriate amount of pharmaceutically acceptable carrier or excipients, sitologically acceptable additive, or topically acceptable carrier such as distilled water, buffer, or isotonic solution, if necessary, with an appropriate amount of the other additives such as the other antibiotics, dye, flavor etc to obtain the inventive composition.

Accordingly, in an another embodiment of the present invention, the present invention provides a method for preparing the inventive cleansing combination comprising the step: of preparing a refined salt, processed salt such as melted salt, or an un-refined salt such as sea salt, rock-salt etc, preferably, refined salt or processed salt prepared by melting un-refined salt at the temperature ranging from 200 to 2000° C., preferably, from 800 to 1200° C., for the period ranging from 2 hours to 7 days, preferably, 12 hours to 48 hours to obtain the melted salt at the 1^{st} step; mixing the salt with sugar compounds such as mono-saccharides, disaccharides, oligosaccharide, polysaccharide etc and lactic acid bacteria belonged to Lactobacillus spp., Bifidobacterium spp., Bacillus spp., Streptococcus spp., Enterococcus spp. Saccharomyces spp., Leuconostoc spp. etc with mixed ratio of mixed ratio of 1:100-0.01:100-0.01 weight part (w/w), preferably, 1:50-0.5:50-0.5 weight part (w/w), more preferably, 1:30-0.3:30-0.3 weight part (w/w), more and more preferably, 1:10-0.1:10-0.1 weight part (w/w), most preferably, 1:5-1:5-1 weight part (w/w) to obtain inventive combination at the 2^{nd} step; and dissolving the combination in an appropriate amount of distilled water, buffer, or isotonic solution, if necessary, with an appropriate amount of the other additives such as the other antibiotics, dye, flavor etc at the 3^{rd} step to obtain the inventive cleansing composition.

The term, “vaginosis” defined herein comprise a vaginosis selected from bacterial vaginosis, fungal vaginitis or Tricomonas vaginitis, preferably, a vaginosis caused Gardnerella vaginalis, Bacterioid fragilis, Tricomonas vaginalis, Candida albicans, Streptococcus agalactiae, Streptococcus aureus, Staphylococcus aureus, Neisseria gonorrhoeae, Escherichia coli, Enterobacter cloacae, Pseudomonas aeruginosa, or Salmonella typhimurium, and the like.

The composition of the present invention may further contain the other antibiotics, dye, flavor etc in the amount of about 0.1˜20% by weight of the above composition based on the total weight of the composition.

It have been proved that the inventive composition comprising a combination of salt, sugar and lactic acid bacteria prepared by the above-described method showed potent antibacterial activity through various experiments, for example, (1) the indirect inhibitory activity test from the growth of vaginosis causing bacteria by determining the change of pH and lactic acid level (Experimental example 1); (2) the direct inhibitory activity test from the growth of vaginosis causing bacteria by determining the susceptibility of test sample (Experimental example 2); (3) brief clinical tests, and finally confirmed that the combination showed potent antibacterial activity in the test.

Accordingly, inventive composition comprising a combination of salt, sugar and lactic acid bacteria prepared by the above-described method for treating or preventing vaginosis, together with a pharmaceutically acceptable carrier.

Additionally, the present invention provides a use of a combination of salt, sugar and lactic acid bacteria prepared by the above-described method in the manufacture of a medicament employed for treating or preventing vaginosis disease in a mammal.

Additionally, the present invention provides a method of treating or preventing vaginosis disease in a mammal wherein method comprises administering to said mammal an effective amount of a combination of salt, sugar and lactic acid bacteria prepared by the above-described method, together with a pharmaceutically acceptable carrier thereof.

The term “prevent” defined herein means the inhibition of such those diseases in a mammal which is prone to be caught by those disease and the term “treat” used herein means (a) the inhibition of the development of disease or illness; (b) the alleviation of disease or illness; or (c) the elimination of disease or illness.

The term “pharmaceutically acceptable carriers or excipients” defined herein comprises “pharmaceutical additives, the inactive ingredients used to make up a medication. They include dyes, flavors, binders, emollients, fillers, lubricants, preservatives, and many more classifications. Common excipients include cornstarch, lactose, talc, magnesium stearate, sucrose, gelatin, calcium stearate, silicon dioxide, shellac and glaze, which has been well-known in the art (See, Home-page of Food and Drug Administration or drug information online) or previous literature (for example, Rowe, Raymond C et al., Handbook of Pharmaceutical Excipients, Pharmaceutical Press, 7th Edition, 2012)

The inventive composition for treating and preventing vaginosis disease may comprises above combination as 0.1˜99%, preferably, 0.1˜50% by weight based on the total weight of the composition.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds. The desirable dose of the inventive combination varies depending on the condition and the weight of the subject, severity, drug form, route and period of administration, and may be chosen by those skilled in the art. However, in order to obtain desirable effects, it is generally recommended to administer at the amount ranging from 0.0001 to 1000 mg/kg, preferably, 0.001 to 100 mg/kg by weight/day of the inventive combination of the present invention. The dose may be administered in single or divided into several times per day.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

The inventive composition of the present invention also can be used as a main component or additive and aiding agent in the preparation of various health functional food and health care food.

Accordingly, it is the other object of the present invention to provide a health functional food comprising a salt, sugar and lactic acid bacteria for the prevention or alleviation of vaginosis disease.

The term “a health functional food” defined herein” the functional food having enhanced functionality such as physical functionality or physiological functionality by adding the composition of the present invention to conventional food to prevent or improve the purposed diseases in human or mammal.

It is the other object of the present invention to provide a health care food comprising a salt, sugar and lactic acid bacteria, together with a sitologically acceptable additive for the prevention or alleviation of vaginosis disease.

The term “a health care food” defined herein “the food containing the combination of the present invention showing no specific intended effect but general intended effect in a small amount of quantity as a form of additive or in a whole amount of quantity as a form of powder, granule, capsule, pill, tablet etc.

The term “a sitologically acceptable additive” defined herein comprises “any substance the intended use which results or may reasonably be expected to result—directly or indirectly—in its becoming a component or otherwise affecting the characteristics of any food”, and can be classified into three groups according to its origin, i.e., (1) chemically synthetic additive such as ketones, glycine, potassium citrate, nicotinic acid, etc; (2) natural additive such as persimmon dye, licorice extract, crystalline cellulose, gua gum etc; (3) the mixed additive therewith such as sodium L-glutamate, preservatives, tar dye etc, or various categories according to its function in the food, for example, thickening agent, maturing agent, bleaching agent, sequestrant, humectant, anti-caking agent, clarifying agents, curing agent, emulsifier, stabilizer, thickener, bases and acid, foaming agents, nutrients, coloring agent, flavoring agent, sweetener, preservative agent, anti-oxidant, etc, which has been well-known in the art or previous literature (See, “Codex General Standard for Food Additives” (GSFA, Codex STAN 192-1995) in Home-page of GSFA Online).

If a substance is added to a food for a specific purpose in that food, it is referred to as a direct additive and indirect food additives are those that become part of the food in trace amounts due to its packaging, storage or other handling.

The term “health care foods or health functional foods” disclosed herein can be contained in food, health beverage, dietary supplement etc, and may be formulated into a form of pharmaceutically dosing form such as a powder, granule, tablet, suspension, emulsion, syrup, chewing tablet, capsule, beverage etc; or the food form, for example, bread, rice cake, dry fruit, candy, chocolate, chewing gum, ice cream, milk such as low-fat milk, lactose-hydrolyzed milk, goat-milk, processed milk, milk product such as fermented milk, butter, concentrated milk, milk cream, butter oil, natural cheese, processed cheese, dry milk, milk serum etc, processed meat product such as hamburger, ham, sausage, bacon etc, processed egg product, fish meat product such as fish cake etc, noodle products such as instant noodles, dried noodles, wet noodles, fried noodles, non-fried noodles, gelatinized dry noodles, cooked noodles, frozen noodles, Pasta etc, tea product such as tea bag, leached tea etc, health drinks such as fruit drinks, vegetable drinks, carbonated soft drinks, soymilk drinks, lactic beverage mixed beverage, etc, seasoning food such as soy sauce, soybean paste, red pepper paste, chunjang (a kind of fermented soybean product colored by caramel), cheonggukjang (natural fermented soybean by B. subtillis), mixed paste, vinegar, sauce, ketchup, curry, dressing etc, margarine, shortening, pizza etc, but not intended herein to limit thereto, for preventing or improving of purposed disease.

Also, above described combination can be added to food or beverage for prevention and improvement of purposed disorder. The amount of above described combination in food or beverage as a functional health food or health care food may generally range from about 0.01 to 100 w/w % of total weight of food for functional health food composition. In particular, although the preferable amount of the combination of the present invention in the functional health food, health care food or special nutrient food may be varied in accordance to the intended purpose of each food, it is preferably used in general to use as an additive in the amount of the combination of the present invention ranging from about 0.01 to 5% in food such as noodles and the like, from 40 to 100% in health care food on the ratio of 100% of the food composition.

Providing that the health beverage composition of present invention contains above combination as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartame et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese, chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition. Examples of addable food comprising aforementioned extract or compound therein are various food, beverage, gum, vitamin complex, health improving food and the like.

Inventive combination of the present invention has no toxicity and adverse effect therefore; they can be used with safe.

It is the other object of the present invention provides a topical composition comprising a combination of salt, sugar and lactic acid bacteria as an active ingredient to treat or prevent vaginosis.

The inventive topical composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as an inventive topical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the present invention may be formulated so as to provide quick, sustained or delayed release of the active ingredient after their administration to a patient by employing any of the procedures well known in the art.

For example, the topical compositions of the present invention can be dissolved in distilled water, pH buffer, oils, propylene glycol or other solvents that are commonly used in the art. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

The inventive composition of the present invention may be prepared in any form, for example, topical preparation such as a aqueous solution, non-aqueous solvent, suspension, emulsion, lyophilized preparation, suppository preparation, cleansing liquid, gel, jelly, foam, cream, ointment, lotion, balm, patch, paste, spray solution, aerosol and the like, or insert preparation such as vaginal tablet, vaginal cream, vaginal ointment, dressing solution, spraying preparation, vaginal capsule, vaginal film, vaginal sponge, spreading or spraying preparation for hygienic goods such as a tampon, sanitary pad, diaper, panties etc, preferably, vaginal tablet composition or cleansing liquid composition, which be added with dissolving adjuvant, emulsifier, pH controller etc.

The above non-aqueous solvent and suspension may comprise propylene glycol, polyethylene glycol, vegetable oil such as olive oil, ethyl olate and the like.

The above suppository preparation may comprise witepsol, macrogol, tween 61, kakao butter, lauric acid, glycerol gelatin and the like.

Accordingly, the present invention provides a cleansing liquid solution or vaginal tablet composition comprising a combination of salt, sugar and lactic acid bacteria for treating or preventing vaginosis, together with a pharmaceutically acceptable carrier.

The topical composition of the present invention in pharmaceutical dosage forms may be used in the form of their pharmaceutically acceptable salts, and also may be used alone or in appropriate association, as well as in combination with other pharmaceutically active compounds such as antibacterial compounds or extract derived from plant, animal or mineral well-known in the art.

The desirable dose of the inventive combination of the present invention varies depending on the condition and the weight of the subject, severity, drug form, route and period of administration, and may be chosen by those skilled in the art. However, in order to obtain desirable effects, it is generally recommended to administer at the amount ranging 0.001-1000 mg/kg, preferably, 0.01 to 100 mg/kg by weight/day of the combination of the present invention. The dose may be administered in single or divided into several times per day. In terms of composition, the inventive combination should be present between 0.01 to 99.99% by weight, preferably 0.1 to 99%, more preferably, 1 to 20%, most preferably, 5 to 10% by weight based on the total weight of the composition.

The composition of present invention can be administered to a subject animal such as mammals (rat, mouse, domestic animals or human) via various routes. All modes of administration are contemplated, for example, administration can be made externally, topically, orally, rectally or by intravenous, intramuscular, subcutaneous, intracutaneous, intrathecal, epidural or intracerebroventricular injection, preferably, externally or topically.

It is the other object of the present invention provides a detergent composition comprising a combination of salt, sugar and lactic acid bacteria as an active ingredient to alleviate or prevent vaginosis.

Any traditional cleaning ingredients can be used as part of the compositions of invention. The levels given are weight percent and refer to the total composition (excluding the water-soluble film in the case of enveloped composition executions). The detergent compositions can be built or unbuilt and comprise one or more detergent active components which may be selected from bleach, bleach activator, bleach catalyst, surfactants, alkalinity sources, enzymes, polymeric dispersants, anti-corrosion agents (e.g. sodium silicate) and care agents. Highly preferred detergent components include a builder compound, an alkalinity source, an anti-redeposition agent, a sulfonated polymer, an enzyme and an additional bleaching agent.

The inventive detergent composition of the present invention may be prepared in any form, for example, topical preparation such as a aqueous solution, non-aqueous solvent, suspension, emulsion, lyophilized preparation, suppository preparation, cleansing liquid, gel, jelly, foam, cream, ointment, lotion, balm, patch, paste, spray solution, aerosol and the like, or insert preparation such as vaginal tablet, vaginal cream, vaginal ointment, dressing solution, spraying preparation, vaginal capsule, vaginal film, vaginal sponge, spreading or spraying preparation for hygienic goods such as a tampon, sanitary pad, diaper, panties etc, preferably, vaginal tablet composition or cleansing liquid composition, which be added with dissolving adjuvant, emulsifier, pH controller etc.

Builders suitable for use herein include builder which forms water-soluble hardness ion complexes (sequestering builder) such as citrates and polyphosphates e.g. sodium tripolyphosphate and sodium tripolyphosphate hexahydrate, potassium tripolyphosphate and mixed sodium and potassium tripolyphosphate salts and builder which forms hardness precipitates (precipitating builder) such as carbonates e.g. sodium carbonate.

Products in unit dose form include tablets, capsules, sachets, pouches, etc

It will be apparent to those skilled in the art that various modifications and variations can be made in the compositions, use and preparations of the present invention without departing from the spirit or scope of the invention.

### Advantageous Effects

As described in the present invention, the inventive composition comprising a combination of salt, sugar and lactic acid bacteria prepared by the above-described method showed potent antibacterial activity through various experiments, for example, (1) the indirect inhibitory activity test from the growth of vaginosis causing bacteria by determining the change of pH and lactic acid level (Experimental example 1); (2) the direct inhibitory activity test from the growth of vaginosis causing bacteria by determining the susceptibility of test sample (Experimental example 2); (3) brief clinical tests, and finally confirmed that the combination showed potent antibacterial activity in the test. Accordingly, the inventive combination may be useful to alleviate, treat or prevent vaginosis in the form of a pharmaceutical composition, health functional food, food additive, topical composition, and detergent composition

### BEST MODE

### Best Mode for Carrying Out the Invention

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Reference Example, Examples and Experimental Examples are intended to further illustrate the present invention without limiting its scope.

### Example 1. Preparation of an Inventive Combination

### 1-1. Preparation of Salt

### 1-1-1. Preparation of Melted Salt

900 mg of un-refined salt (Shinan salt coop, 88-6, Dongniseonchakjang-gil, Sangtaedong-ri, Sinui-myeon, Sinan-gun, Jeollanam-do, Republic of Korea, 58857) was melted for 24 hours at 850-1000° C. using by heater (MS-E104, TOPS Co. Ltd.) to obtain 400 mg of the melted salt (designated as “MS” hereinafter).

### 1-1-2. Preparation of Refined Salt

400 mg of refined salt (NaCl, F.W. 58.44) was procured the company (SPPO-91701, Duksan Pure Chemicals, Co., Ltd, 53, Siwon-ro, 133beon-gil, Danwon-gu, Ansan-si, Gyeonggi-do, Korea) (designated as “RS” hereinafter).

### 1-1-3. Preparation of Unrefined Salt

400 mg of un-refined salt (natural salt) was procured the company (Shinan salt coop, 88-6, Dongniseonchakjang-gil, Sangtaedong-ri, Sinui-myeon, Sinan-gun, Jeollanam-do, Republic of Korea, 58857) (designated as “US” hereinafter).

### 1-2. Preparation of Sugar

### 1-2-1. Preparation of Glucose

800 mg of glucose (D(+)-glucose) was procured the company (Cat. No. jb01, J. T. Baker Chemical Company). (designated as “GL” hereinafter).

### 1-2-2. Preparation of Fructo-Oligosaccharide

800 mg of fructo-oligosaccharide (derived from chicory) was procured the company (Cat. No. F8052050 g, Sigma-Aldrich Co. LLC). (designated as “FO” hereinafter).

### 1-2-3. Preparation of Lactulose

800 mg of lactulose was procured the company (Cat. No., 126-03732, Wako Pure Chemical Industries). (designated as “LS” hereinafter).

### 1-2-4. Preparation of Fructose

800 mg of fructose was procured the company (Cat. No., 64505-0410, Junsei Chemical Co. Ltd). (designated as “FS” hereinafter).

### 1-2-5. Preparation of Lactitol

800 mg of lactitol was procured the company (Cat. No., sc488686, Santa Cruz Biotechnology, Inc). (designated as “LT” hereinafter).

### 1-3. Preparation & Culture of Lactic Acid Bacteria

### 1-3-1. Preparation of Lactic Acid Bacteria

Various lactic acid bacteria listed in Table 1 were apportioned from KCTC (Korean Collection for Type Cultures, Korea) and used in the following experiments.

TABLE 1
Lactic Acid Bacteria

| Name | Scientific name | Deposit No. |
|---|---|---|
| BLA | Lactobacillus acidophilus | KCTC No. 3164 |
| BLB | Lactobacillus brevis | KCTC No. 13094 |
| BLC | Lactobacillus casei | KCTC No. 13086 |
| BLD | Lactobacillus delbrueckii subsp. bulgaricus | KCTC No. 3635 |
| BLLC | Lactobacillus casei subsp. casei | KCTC No. 3260 |
| BLDD | Lactobacillus delbrueckii subsp. delbrueckii | KCTC No. 13721 |
| BBL | Bifidobacterium longum sub sp. longum | KCTC No. 3128 |
| BBA | Bifidobacterium adolescentis | KCTC No. 3267 |
| BBB1 | Bifidobacterium bifidum | KCTC No. 3357 |
| BBB2 | Bifidobacterium breve | KCTC No. 3441 |
| BBC2 | Bifidobacterium catenulatum | KCTC No. 3221 |
| BBC | Bacillus cereus | KCTC No. 13123 |
| BSS | Streptococcus sp. | KCTC No. 5644 |

### 1-3-2. Culture of Bacteria

(1) Facultative anaerobic and microaerophilic bacteria, i.e., Lactobacillus acidophilus (KCTC No. 3164), Streptococcus sp. (KCTC No. 5644), Bifidobacterium longum sub sp. longum (KCTC No. 3128) etc and aerobic bacteria, i.e., Bacillus cereus (KCTC No. 13123) etc were used in the experiment.

(2) In addition to the above lactic acid bacteria, vaginosis causing bacteria, i.e., Bacterioides fragilis (KCTC No. 5013) and Gardnerella vaginalis (KCTC No. 5097) were used in the experiment.

(3) The vaginosis causing bacteria, i.e., Bacterioides fragilis (KCTC No. 5013) and Gardnerella vaginalis (KCTC No. 5097) were inoculated into Casmans medium supplemented with 5% sheep blood (12 g of Tryptone, 5 g of meat peptone, 5 g of sodium chloride, 3 g of yeast extract, 3 g of beef extract, 1 g of starch casein, 0.5 g of D-glucose, 0.05 g of nicotinamide, 0.005 g of p-aminobenzoic acid, 1 L of distilled water, pH 7.3) and performed to shaking incubation at 37° C. at the speed of 150 rpm using by GasPak TMEZ Pouch system (BD, Cat. No. 26083, USA).

(4) Facultative anaerobic and microaerophilic bacteria, i.e., Lactobacillus acidophilus (KCTC No. 3164), Streptococcus sp. (KCTC No. 5644), Bifidobacterium longum sub sp. longum (KCTC No. 3128) etc and aerobic bacteria, i.e., Bacillus cereus (KCTC No. 13123) etc were inoculated into MRS medium (10 g of Protease peptone, 10 g of beef extract, 5 g of yeast extract, 20 g of D-glucose, 1 ml of Tween 80, 2 g of K₂HPO₄, 5 g of sodium acetate, 2 g of diammonium hydrogencitrate, 0.2 g of MgSO₄₇H₂0, 0.2 g of MnSO₄H₂0, 1 L of distilled water, pH 6.2-6.5) and performed to shaking incubation at 37° C. at the speed of 150 rpm.

### 1-4. Preparation of Inventive Combination

50 mg of the melted salt prepared in the above step was thoroughly mixed with 5 mg of dried glucose and 1 mg of dried Lactobacillus acidophilus prepared in the above steps, together to obtain the inventive combination (designated as “CL1” hereinafter).

The combinations were kept at −75° C. in refrigerator and used in following experiments by dissolving in distilled water before use.

The other combinations of the present invention listed in Table 2 were prepared according to the similar method to the above.

TABLE 2
Combinations

| Group | A. | B. | C. |
|---|---|---|---|
| Name | salt* | sugar** | lactic acid bacteria*** |
| CL1 | RS (5 mg) | GL (5 mg) | BLA (1 mg) |
| CL2 | MS (5 mg) | FO (10 mg) | BLB (1 mg) |
| CL3 | US (5 mg) | LS (25 mg) | BLC (1 mg) |
| CL4 | RS (5 mg) | FS (2.5 mg) | BLD (1 mg) |
| CL5 | MS (5 mg) | LT (1 mg) | BLLC (1 mg) |
| CB1 | RS (5 mg) | GL (5 mg) | BBC (1 mg) |
| CB2 | MS (5 mg) | FO (10 mg) | BBC (1 mg) |
| CB3 | US (5 mg) | LS (25 mg) | BBC (1 mg) |
| CB4 | RS (5 mg) | FS (2.5 mg) | BBC (1 mg) |
| CB5 | MS (5 mg) | LT (1 mg) | BBC (1 mg) |
| CF1 | RS (5 mg) | GL (5 mg) | BBL (1 mg) |
| CF2 | MS (5 mg) | FO (10 mg) | BBA (1 mg) |
| CF3 | US (5 mg) | LS (25 mg) | BBB1 (1 mg) |
| CF4 | RS (5 mg) | FS (2.5 mg) | BBB2 (1 mg) |
| CF5 | MS (5 mg) | LT (1 mg) | BBC2 (1 mg) |
| CS1 | RS (5 mg) | GL (5 mg) | BSS (1 mg) |
| CS2 | MS (5 mg) | FO (10 mg) | BSS (1 mg) |
| CS3 | US (5 mg) | LS (25 mg) | BSS (1 mg) |
| CS4 | RS (5 mg) | FS (2.5 mg) | BSS (1 mg) |
| CS5 | MS (5 mg) | LT (1 mg) | BSS (1 mg) |
| CP1 | — | — | BLA (1 mg) |
| CP2 | — | — | BBC (1 mg) |
| CP3 | — | — | BBL (1 mg) |
| CP4 | — | — | BSS (1 mg) |

*: RS (Refined salt), MS (melted salt), US (Unrefined salt)
**: GL (glucose), FO (Fructo-oligosaccharide), LS (lactulose), FS (Fructose), LT (lactitol)
***: BLA (Lactobacillus acidophilus), BLB (Lactobacillus brevis), BLC (Lactobacillus casei), BLD (Lactobacillus delbrueckii subsp. bulgaricus), BLLC (Lactobacillus casei subsp. Casei), BLDD (Lactobacillus delbrueckii subsp. Delbrueckii), BBL (Bifidobacterium longum subsp. Longum), BBA (Bifidobacterium adolescentis), BBB1 (Bifidobacterium bifidum), BBB2 (Bifidobacterium breve), BBC2 (Bifidobacterium catenulatum), BBC (Bacillus cereus), BSS (Streptococcus sp)

### Example 2. Preparation of Inventive Vaginal Tablet Composition

The combination prepared in Example 1 comprising 400 mg of melted salt, 800 mg of glucose and 40 mg of dried Lactobacillus acidophilus was mixed with 2 mg of magnesium stearate in order to formulating into inventive vaginal tablet composition combination (designated as “SGL2” hereinafter) using by entableting apparatus (KT2000, Kumsungkigong).

### Example 3. Preparation of Inventive Vaginal Cleansing Solution Composition

The vaginal cleansing solution composition comprising the combination prepared in Example 1 comprising 400 mg of refined salt, 800 mg of glucose and 40 mg of dried Bifidobacterium longum sub sp. longum was prepared by mixing with following ingredients as shown in Tablet 3 (designated as “SGL3” hereinafter) for 48 hours with stirring.

TABLET 3
SGL4 solution (100 ml)

| Ingredient | | Amount | |
|---|---|---|---|
| SGL3 | | 0.5 | g |
| Lactic acid | | 1 | g |
| adjuvant | Whey | 180 | mg |
| | Ethanol | 1 | g |

| | |
|---|---|
| Preservatives | Trace amount |
| (benzalkonimum HCl and menthol) | |

| | |
|---|---|
| Distilled water | Appropriate amount to adjusted to 100 ml |

### Experimental Example 1. Inhibition Effect on the Growth of Vaginosis Causing Strains

To determine the inhibitory effect of inventive combinations prepared in Example 1 on the growth of vaginosis-causing strains indirectly, following test was performed according to the procedure disclosed in the literature (Choi, J. G. et al, Antibacterial activity of Ecklonia cava against methicillin-resistant Staphylococcus aureus and Salmonella spp., Foodborne Pathog. Dis., 2010 (April): 7(4), pp 435-441).

### 1-1. Testing Strains

The vaginosis causing bacteria, i.e., Bacterioides fragilis (KCTC No. 5013) and Gardnerella vaginalis (KCTC No. 5097) were inoculated into Casmans medium supplemented with 5% FBS (Fetal bovine Serum) (12 g of Tryptone, 5 g of meat peptone, 5 g of sodium chloride, 3 g of yeast extract, 3 g of beef extract, 1 g of starch casein, 0.5 g of D-glucose, 0.05 g of nicotinamide, 0.005 g of p-aminobenzoic acid, 1 L of distilled water, pH 7.3, 5% FBS) and performed to shaking incubation at 37° C. at the speed of 150 rpm using by GasPak™EZ Pouch system (Anaerobic Gas Generation Pouch System with indicator, BD, Cat. No. 26083, USA) according to the cited literature (D. D. Charles, et al., Identification of haemophilus vaginalis, 1960, Journal of bacteriology p277, R. P. Morgan et al., Amniotic fluid and maternalrace influence responsiveness of fetal membranes to bacteria, Journal of reproductive immunology, 96 (2012) 68-78).

### 1-2. Experimental Materials

The inventive combinations shown in Table 2 were used as test samples and lactic acid was purchased from the company (Cat. No. 252476, ACS reagent, 85-90%, L-lactic acid in water, Sigma-Aldrich Co. LLC).

### 1-3. Purpose of Experiment

The purpose of experiment is to determine the change of OD (optical density), pH and level of lactic acid in the test group treated with test samples prepared in Example 1 comparing with control group treated with only vaginosis-causing strains.

### 1-4. Experimental Procedure

### 1-4-1. Control Group

The test samples prepared in Example 1 were added to the culturing medium of vaginosis-causing strains, i.e., Bacterioides fragilis (KCTC No. 5013) and Gardnerella vaginalis (KCTC No. 5097). The pre-cultured vaginosis-causing strains (Absorbance: OD₆₆₀=1.0 A, 1/100 v/v) were inoculated into Casman medium containing FBS again and performed to shaking incubation at 37° C. at the speed of 150 rpm for 12 hours. The control group was treated with test samples prepared in Example 1

### 1-4-2. Comparative Group

Various lactic acid bacteria as shown in Table 1, were only added to the culturing medium of vaginosis-causing strains, i.e., Bacterioides fragilis (KCTC No. 5013) and Gardnerella vaginalis (KCTC No. 5097). The pre-cultured vaginosis-causing strains (Absorbance: OD₆₆₀=1.0 A, 1/100 v/v) were inoculated into Casman medium containing FBS again and performed to shaking incubation at 37° C. at the speed of 150 rpm for 12 hours. The comparison group treated with only lactic acid bacteria.

### 1-4-3. Test Sample Group

The combination of 1 mg of lactic acid bacteria and the test samples prepared in Example 1 were added to the culturing tube of vaginosis-causing strains, i.e., Bacterioides fragilis (KCTC No. 5013) and Gardnerella vaginalis (KCTC No. 5097) and performed to shaking incubation at 37° C. at the speed of 150 rpm for 18 hours. The test sample group treated with 1 mg of lactic acid bacteria and the test samples prepared in Example 1.

### 1-4-4. Determination of pH Value

The change of pH value in the control group and test sample groups was determined as follows.

The pH of cultured cell solution in the control group and test sample groups was determined using by apparatus (pH meter, SevenEasy, Mettler Toledo, Swiss).

### 1-4-5. Determination of the Level of Lactic Acid

The level of lactic acid in the control group and test sample groups was determined using by assay kit (D-/L-Lactic acid (Rapid) Assay kit, megazyme, Cat. No. K-DLATE) as follows.

1.5 ml of distilled water, 0.1 ml of test sample and 0.02 ml of buffer solution (Buffer; plus D-glutamate and sodium azide (0.02% w/v), 0.1 ml of NAD, DGP (D-Glutamate-pyruvate transaminase suspension) were poured to cuvettes and mixed together for 3 mins. The absorbance of A1 value (wavelength: 340 nm) is determined using by spectrophotometer (Spectronic Genesys 2, Thermo, USA) and 0.02 ml of D-LDL (D-Lactate dehydrogenase suspension, D-/L-lactic acid kit, Megazyme, Ireland) was added to the solution to mix together for 5 mins.

The absorbance of A2 value (wavelength: 340 nm) is determined using by spectrophotometer (Spectronic Genesys 2, Thermo, USA).

Finally, 0.02 ml of D-LDL (D-Lactate dehydrogenase suspension, D-/L-lactic acid kit, Megazyme, Ireland) was added to the solution to mix together for 10 mins and the absorbance of A3 value (wavelength: 340 nm) is determined using by spectrophotometer (Spectronic Genesys 2, Thermo, USA).

The level of lactic acid in the control group and test sample groups was determined using by software (Mega-Calc™, Megazyme, IDA Business Park, Bray, Co. Wicklow, A98 YV29, Ireland) and the resulting absorbance values of A1-A3.

### 1-5. Test Result

The test results on the change of OD (optical density), pH and level of lactic acid in the test group treated with test samples prepared in Example 1 comparing with control group treated with only vaginosis-causing strains were shown in Tables 4 & 5 (Gadnerella vaginalis strain) and Tables 6 & 7 (Bacterioides fragilis strain).

1-5-1. Gardnerella vaginalis Strain (See Tables 4 & 5)

In case of the experiment treated to Gardnerella vaginalis strain, the test group treated with inventive combinations showed acidic environment, i.e., pH range of 4.0 to 4.9, providing with the inhibiting environment from the growth of Gardnerella vaginalis strain in vagina whereas the control group without inventive combinations showed pH range of 5.19 to 5.52.

As to level of lactic acid having inhibiting activity from the growth of detrimental bacteria in vagina, the level of lactic acid in the test groups treated with inventive combinations ranged 0.970 mg/ml to 1.712 mg/ml, providing with more potent inhibiting activity from the growth of Gardnerella vaginalis strain in vagina compared with the comparative group treated with only lactic acid bacteria whereas the control group without inventive combinations showed the level of lactic acid ranging from 0.711 mg/ml to 0.765 mg/ml.

1-5-2. Bacterioides fragilis Strain (See Tables 6 & 7)

In case of the experiment treated to Bacterioides fragilis strain, the test group treated with inventive combinations showed acidic environment, i.e., pH range of 4.4 to 4.9, providing with the inhibiting environment from the growth of Bacterioides fragilis strain in vagina whereas the control group without inventive combinations showed pH range of 5.21 to 5.62.

As to level of lactic acid having inhibiting activity from the growth of detrimental bacteria in vagina, the level of lactic acid in the test groups treated with inventive combinations ranged 0.4420 mg/ml to 1.049 mg/ml, providing with more potent inhibiting activity from the growth of Bacterioides fragilis strain in vagina compared with the comparative group treated with only lactic acid bacteria whereas the control group without inventive combinations showed the level of lactic acid of 0.000 mg/ml.

Accordingly, it has been confirmed that the inventive combinations promoted the growth of lactic acid bacteria by way of providing with necessary nutrients, resulting in stimulating the reproduction of lactic acid maintaining a vagina with an acidic environment.

TABLE 4
Change of pH and level of lactic acid (Gardnerella vaginalis strain)

| Gardnerella vaginalis | Lactic acid bacteria* | | | |
|---|---|---|---|---|
| Gardnerella vaginalis | BLA | BBC | BBL | BSS |
| Control group (media pH = 7.3) | 61 Ctrl. | 62 Ctrl. | 63 Ctrl. | 64 Ctrl. |
| Control group | pH = 5.45 | pH = 5.52 | pH = 5.45 | pH = 5.19 |
| (media pH = 7.3) | Lactate** | Lactate** | Lactate** | Lactate** |
| | (mg/ml) = 0.762 | (mg/ml) = 0.711 | (mg/ml) = 0.751 | (mg/ml) = 0.711 |
| | (D:0.016/L:0.746) | (D:0.015/L:0.696) | (D:0.014/L:0.737) | (D:0.014/L:0.697) |
| Test group I | CL1 | CB1 | CF1 | CS1 |
| Test group I | pH = 4.32 | pH = 4.65 | pH = 4.22 | pH = 4.26 |
| | Lactate** | Lactate** | Lactate** | Lactate** |
| | (mg/ml) = 1.052 | (mg/ml) = 1.022 | (mg/ml) = 1.061 | (mg/ml) = 1.025 |
| | (D:1.052/L:0.000) | (D:0.014/L:1.008) | (D:0.017/L:1.044) | (D:0.014/L:1.011) |
| Test group II | CL2 | CB2 | CF2 | CS2 |
| Test group II | pH = 5.01 | pH = 5.18 | pH = 5.22 | pH = 5.07 |
| | Lactate** | Lactate** | Lactate** | Lactate** |
| | (mg/ml) = 1.005 | (mg/ml) = 0.970 | (mg/ml) = 0.758 | (mg/ml) = 1.712 |
| | (D:0.218/L:0.787) | (D:0.017/L:0.953) | (D:0.016/L:0.742) | (D:0.014/L:0.673) |
| Comparative group (media pH = 7.3) | CP1 | CP2 | CP3 | CP4 |
| Comparative group | pH = 5.43 | pH = 5.55 | pH = 5.47 | pH = 5.22 |
| (media pH = 7.3) | Lactate** | Lactate** | Lactate** | Lactate** |
| | (mg/ml) = 0.753 | (mg/ml) = 0.709 | (mg/ml) = 0.754 | (mg/ml) = 0.715 |
| | (D:0.015/L:0.738) | (D:0.011/L:0.698) | (D:0.015/L:0.739) | (D:0.013/L:0.702) |

*BLA (Lactobacillus acidophilus), BBC (Bacillus cereus), BBL (Bifidobacterium longum subsp. Longum), BSS (Streptococcus sp);
**D; D-LDL/L; L-LDL

TABLE 5
Change of pH and level of lactic acid (Gardnerella vaginalis strain)

| Gardnerella vaginalis | Lactic acid bacteria* | | | |
|---|---|---|---|---|
| Gardnerella vaginalis | BLA | BBC | BBL | BSS |
| Control group (media pH = 7.3) | 61 Ctrl. | 62 Ctrl. | 63 Ctrl. | 64 Ctrl. |
| Control group | pH = 5.45 | pH = 5.52 | pH = 5.45 | pH = 5.19 |
| (media pH = 7.3) | Lactate** | Lactate** | Lactate** | Lactate** |
| | (mg/ml) = 0.762 | (mg/ml) = 0.711 | (mg/ml) = 0.751 | (mg/ml) = 0.711 |
| | (D:0.016/L:0.746) | (D:0.015/L:0.696) | (D:0.014/L:0.737) | (D:0.014/L:0.697) |
| Test group III | CL3 | CB3 | CF3 | CS3 |
| Test group III | pH = 4.27 | pH = 4.99 | pH = 4.20 | pH = 5.09 |
| | Lactate** | Lactate** | Lactate** | Lactate** |
| | (mg/ml) = 1.046 | (mg/ml) = 0.940 | (mg/ml) = 1.064 | (mg/ml) = 0.665 |
| | (D:0.991/L:0.055) | (D:0.017/L:0.923) | (D:0.021/L:1.043) | (D:0.013/L:0.652) |
| Test group IV | CL4 | CB4 | CF4 | CS4 |
| Test group IV | pH = 4.38 | pH = 4.89 | pH = 4.55 | pH = 4.87 |
| | Lactate** | Lactate** | Lactate** | Lactate** |
| | (mg/ml) = 1.045 | (mg/ml) = 1.017 | (mg/ml) = 1.033 | (mg/ml) = 0.806 |
| | (D:0.764/L:0.281) | (D:0.021/L:0.996) | (D:0.015/L:1.018) | (D:0.013/L:0.793) |
| Test group V | CL5 | CB5 | CF5 | CS5 |
| Test group V | pH = 5.01 | pH = 5.34 | pH = 5.17 | pH = 5.12 |
| | Lactate** | Lactate** | Lactate** | Lactate** |
| | (mg/ml) = 1.006 | (mg/ml) = 0.942 | (mg/ml) = 0.737 | (mg/ml) = 0.693 |
| | (D:0.237/L:0.767) | (D:0.019/L:0.923) | (D:0.014/L:0.723) | (D:0.013/L:0.680) |
| Comparative group (media pH = 7.3) | CP1 | CP2 | CP3 | CP4 |
| Comparative group | pH = 5.43 | pH = 5.55 | pH = 5.47 | pH = 5.22 |
| (media pH = 7.3) | Lactate** | Lactate** | Lactate** | Lactate** |
| | (mg/ml) = 0.753 | (mg/ml) = 0.709 | (mg/ml) = 0.754 | (mg/ml) = 0.715 |
| | (D:0.015/L:0.738) | (D:0.011/L:0.698) | (D:0.015/L:0.739) | (D:0.013/L:0.702) |

*BLA (Lactobacillus acidophilus), BBC (Bacillus cereus), BBL (Bifidobacterium longum subsp. Longum), BSS (Streptococcus sp);
**D; D-LDL/L; L-LDL

TABLE 6
Change of pH and level of lactic acid (Bacterioides fragilis strain)

| Bacterioides fragilis | Lactic acid bacteria* | | | |
|---|---|---|---|---|
| Bacterioides fragilis | BLA | BBC | BBL | BSS |
| Control group (media pH = 7.3) | 51 Ctrl. | 52 Ctrl. | 53 Ctrl. | 54 Ctrl. |
| Control group | pH = 5.58 | pH = 5.36 | pH = 5.62 | pH = 5.21 |
| (media pH = 7.3) | Lactate** | Lactate** | Lactate** | Lactate** |
| | (mg/ml) = 0.000 | (mg/ml) = 0.000 | (mg/ml) = 0.000 | (mg/ml) = 0.000 |
| | (D:0.000/L:0.000) | (D:0.000/L:0.000) | (D:0.000/L:0.000) | (D:0.000/L:0.000) |
| Test group I | CL1 | CB1 | CF1 | CS1 |
| Test group I | pH = 4.47 | pH = 4.70 | pH = 4.48 | pH = 4.55 |
| | Lactate** | Lactate** | Lactate** | Lactate** |
| | (mg/ml) = 1.049 | (mg/ml) = 0.278 | (mg/ml) = 1.007 | (mg/ml) = 0.324 |
| | (D:1.007/L:0.042) | (D:0.278/L:0.000) | (D:0.327/L:0.680) | (D:0.3244/L:0.000) |
| Test group II | CL2 | CB2 | CF2 | CS2 |
| Test group II | pH = 5.22 | pH = 4.99 | pH = 5.38 | pH = 5.04 |
| | Lactate** | Lactate** | Lactate** | Lactate** |
| | (mg/ml) = 0.451 | (mg/ml) = 0.015 | (mg/ml) = 0.006 | (mg/ml) = 0.004 |
| | (D:0.190/L:0.261) | (D:0.010/L:0.005) | (D:0.005/L:0.001) | (D:0.000/L:0.004) |
| Comparative group (media pH = 7.3) | CP1 | CP2 | CP3 | CP4 |
| Comparative | pH = 5.42 | pH = 5.35 | pH = 5.60 | pH = 5.22 |
| group | Lactate** | Lactate** | Lactate** | Lactate** |
| (media pH = 7.3) | (mg/ml) = 0.002 | (mg/ml) = 0.003 | (mg/ml) = 0.002 | (mg/ml) = 0.004 |
| | (D:0.002/L:0.000) | (D:0.001/L:0.002) | (D:0.001/L:0.001) | (D:0.003/L:0.001) |

*BLA (Lactobacillus acidophilus), BBC (Bacillus cereus), BBL (Bifidobacterium longum subsp. Longum), BSS (Streptococcus sp);
**D; D-LDL/L; L-LDL

TABLE 7
Change of pH and level of lactic acid (Bacterioides fragilis strain)

| Bacterioides fragilis | Lactic acid bacteria* | | | |
|---|---|---|---|---|
| Bacterioides fragilis | BLA | BBC | BBL | BSS |
| Control group (media pH = 7.3) | 51 Ctrl. | 52 Ctrl. | 53 Ctrl. | 54 Ctrl. |
| Control group | pH = 5.58 | pH = 5.36 | pH = 5.62 | pH = 5.21 |
| (media pH = 7.3) | Lactate** | Lactate** | Lactate** | Lactate** |
| | (mg/ml) = 0.000 | (mg/ml) = 0.000 | (mg/ml) = 0.000 | (mg/ml) = 0.000 |
| | (D:0.000/L:0.000) | (D:0.000/L:0.000) | (D:0.000/L:0.000) | (D:0.000/L:0.000) |
| Test group III | CL3 | CB3 | CF3 | CS3 |
| Test group III | pH = 4.45 | pH = 4.80 | pH = 4.85 | pH = 4.73 |
| | Lactate** | Lactate** | Lactate** | Lactate** |
| | (mg/ml) = 1.029 | (mg/ml) = 0.026 | (mg/ml) = 0.116 | (mg/ml) = 0.050 |
| | (D:1.016/L:0.013) | (D:0.023/L:0.003) | (D:0.096/L:0.020) | (D:0.049/L:0.001) |
| Test group IV | CL4 | CB4 | CF4 | CS4 |
| Test group IV | pH = 4.74 | pH = 4.60 | pH = 4.85 | pH = 4.52 |
| | Lactate** | Lactate** | Lactate** | Lactate** |
| | (mg/ml) = 0.939 | (mg/ml) = 0.043 | (mg/ml) = 0.442 | (mg/ml) = 0.514 |
| | (D:0.710/L:0.229) | (D:0.043/L:0.000) | (D:0.432/L:0.010) | (D:0.514/L:0.000) |
| Test group V | CL5 | CB5 | CF5 | CS5 |
| Test group V | pH = 5.30 | pH = 5.03 | pH = 5.37 | pH = 5.03 |
| | Lactate** | Lactate** | Lactate** | Lactate** |
| | (mg/ml) = 0.431 | (mg/ml) = 0.015 | (mg/ml) = 0.011 | (mg/ml) = 0.000 |
| | (D:0.183/L:0.248) | (D:0.012/L:0.003) | (D:0.005/L:0.006) | (D:0.000/L:0.000) |
| Comparative group (media pH = 7.3) | CP1 | CP2 | CP3 | CP4 |
| Comparative | pH = 5.42 | pH = 5.35 | pH = 5.60 | pH = 5.22 |
| group | Lactate** | Lactate** | Lactate** | Lactate** |
| (media pH = 7.3) | (mg/ml) = 0.002 | (mg/ml) = 0.003 | (mg/ml) = 0.002 | (mg/ml) = 0.004 |
| | (D:0.002/L:0.000) | (D:0.001/L:0.002) | (D:0.001/L:0.001) | (D:0.003/L:0.001) |

*BLA (Lactobacillus acidophilus), BBC (Bacillus cereus), BBL (Bifidobacterium longum subsp. Longum), BSS (Streptococcus sp);
**D; D-LDL/L; L-LDL

### Experimental Example 2. Direct Inhibition Effect on the Growth of Vaginosis Causing Strains

To reconfirm the direct inhibitory effect of inventive combinations prepared in Example 1 on the growth of vaginosis-causing strains, following test was performed as follow.

### 2-1. Purpose of the Preliminary Test

The susceptibility of the vaginosis-causing strains and lactic acid bacteria used in the experiment was determined by using various antibiotic-resistance of the vaginosis-causing strains and lactic acid bacteria and the inhibitory activity or promoting effect of the inventive combinations on vaginosis-causing strains can be quantitatively and directly determined.

Various concentrations of various antibiotics, i.e., 50 microgram/ml of ampicillin (Cat. No. AM0510, Georgia Chem. USA), 50 microgram/ml of kanamycin (Cat. No. KA2003, Georgia Chem. USA), 34 microgram/ml of chloramphenicol (Art. 3886.2, Germany), 15 microgram/ml of tetracycline (Art. Nr. HP63.1, Germany), 50 microgram/ml of Streptomycin (Cat. No. S1027, Biosesang, Korea), 20 microgram/ml of gentamycin (Cat. No. GS3007, Georgia Chem. USA) and 25 microgram/ml of rifampicin (Cat. No. R3501, Sigma-Alrich, USA) were used in the experiment.

### 2-2. Preliminary Test on the Susceptibility of the Vaginosis-Causing Strains

### 2-2-1. Experiment Procedure

The vaginosis-causing strains, i.e., Bacterioides fragilis (KCTC No. 5013) and Gardnerella vaginalis (KCTC No. 5097) were inoculated into Casmans medium supplemented with 5% sheep blood and 1.5% agar and performed to shaking incubation for 36 hrs under anaerobic condition using by GasPak TMEZ Gas Generation Pouch system (BD, Cat. No. 26083, USA).

After the incubation, the colony was collected by platinum loop and inoculated to Casmans medium supplemented with 5% FBS (Fetal bovine Serum) (12 g of Tryptone, 5 g of meat peptone, 5 g of sodium chloride, 3 g of yeast extract, 3 g of beef extract, 1 g of starch casein, 0.5 g of D-glucose, 0.05 g of nicotinamide, 0.005 g of p-aminobenzoic acid, 1 L of distilled water, pH 7.3, 5% FBS). The media was performed to shaking incubation at 37° C. for 24 hrs at the speed of 150 rpm using by GasPak™EZ Pouch system (Anaerobic Gas Generation Pouch System with indicator, BD, Cat. No. 26083, USA) according to the cited literatures (Treatment of Gardnella vaginalis infection., J I, Adinma et al., J. Obstetrics and Gynecology, 17(6), pp. 573-575, 1997; Treatment of urinary tract infection by Gardnella vaginalis; a composition of oral metronidazole versus ampicillin, AG. PA., et al., Rev Latioam Microbiol. 2001, 43(2):pp 65-69; Antimicrobial susceptibilities of Gardnella vaginalis., Kharsany A B., et al., antimicrobial agents and chemotherapy, 1993, pp 2733-2735; Susceptibilities of Bacteroides fragilis to six antibiotics determined by standardized antimicrobial disc susceptibility testing., Sutter V L., et al., antimicrobial agents and chemotherapy, 1973, pp 188-193).

### 2-2-2. Test Result

At the result, the antibiotic susceptibility of various antibiotics against vaginosis-causing bacteria was shown in Table 8.

TABLET 8
Antibiotic susceptibility of various antibiotics
against vaginosis-causing bacteria

| | vaginosis-causing bacteria | |
|---|---|---|
| antibiotics | Bacteroides fragilis | Gardnella vaginalis |
| Ampicillin (50 microgram/ml) | +++ | − |
| Kanamycin(50 microgram/ml) | +++ | +++ |
| Chloramphenicol | − | − |
| (34 microgram/ml) | | |
| Tetracyclin (15 microgram/ml) | − | − |
| Streptomycin (50 microgram/ml) | +++ | − |
| Gentamycin (20 microgram/ml) | +++ | +++ |
| Ripampicin (25 microgram/ml) | − | − |

*; +++: being normally grown without the effect of antibiotic,
−: being not grown caused by the effect of antibiotic

### 2-3. Preliminary Test on the Susceptibility of the Lactic Acid Bacteria Strains

### 2-2-1. Experiment Procedure

In order to determine the antibiotic resistance of the lactic acid bacteria strains reproducing abundant lactic acids, i.e., Lactobacillus acidophilus (KCTC No. 3164), Bifidobacterium longum sub sp. longum (KCTC No. 3128) etc, the bacteria was inoculated into MRS medium (10 g of Protease peptone, 10 g of beef extract, 5 g of yeast extract, 20 g of D-glucose, 1 ml of Tween 80, 2 g of K₂HPO₄, 5 g of sodium acetate, 2 g of diammonium hydrogencitrate, 0.2 g of MgSO₄₇H₂0, 0.2 g of MnSO₄H₂0, 1 L of distilled water, pH 6.2-6.5) and performed to shaking incubation at 37° C. at the speed of 150 rpm.

For anaerobic incubation, the media was performed to shaking incubation at 37° C. for 24 hrs at the speed of 150 rpm using by GasPak TMEZ Pouch system (Anaerobic Gas Generation Pouch System with indicator, BD, Cat. No. 26083, USA) according to the cited literatures (Antibiotic susceptibility of members of the lactobacillus acidophilus group using broth microdilution and molecular identification of their resistance determinants, M. Sigrids et al., International Journal of Food Microbiology 144 (2010), pp 81-87; Antibiotic sensitivity of acid stressed probiotic Lactobacillus acidophilus ncdc 291, NNK, GS., The internet Journal of microbiology Vol. 9 (2010); Antimicrobial susceptibility of bifidobactria., F. CM., et al., Journal of antimicrobial Chemotherapy (2005) 55, pp. 38-44).

### 2-2-2. Test Result

At the result, the antibiotic susceptibility of various antibiotics against lactic acid bacteria strains was shown in Table 7.

TABLET 9
Antibiotic susceptibility of various antibiotics against lactic acid
bacteria strains

| | AB* | | | | | | |
|---|---|---|---|---|---|---|---|
| LB** | A50 | K50 | C34 | T15 | S50 | G20 | R25 |
| BLA | −*** | +++ | − | − | − | − | − |
| BLB | − | +++ | − | − | − | − | − |
| BLC | − | +++ | − | − | − | − | − |
| BLD | − | +++ | − | − | − | − | − |
| BBL | − | +++ | − | − | +++ | +++ | − |
| BBA | − | ++ | − | − | ++ | +++ | − |
| BBB1 | − | +++ | − | − | ++ | ++ | − |
| BBB2 | − | +++ | − | − | +++ | ++ | − |
| BBC2 | − | ++ | − | − | ++ | +++ | − |
| BBC | − | ++ | − | − | ++ | +++ | − |
| BSS | − | +++ | − | − | ++ | ++ | − |

*AB (Antibiotics), A50 (Ampicillin, 50 microgram/ml), K50 (Kanamycin, 50 microgram/ml), C34 (Chloramphenicol, 34 microgram/ml), T15 (Tetracyclin, 15 microgram/ml), S50 (Streptomycin, 50 microgram/ml), G20 (Gentamycin, 20 microgram/ml), R25 (Ripampicin, 25 microgram/ml)
**LB (Lactic acid bacteria), BLA (Lactobacillus acidophilus), BLB (Lactobacillus brevis), BLC (Lactobacillus casei), BLD (Lactobacillus delbrueckii subsp. bulgaricus), BBL (Bifidobacterium longum subsp. Longum), BBA (Bifidobacterium adolescentis), BBB1 (Bifidobacterium bifidum), BBB2 (Bifidobacterium breve), BBC2 (Bifidobacterium catenulatum), BBC (Bacillus cereus), BSS (Streptococcus sp),
***+++: being normally grown without the effect of antibiotic, −: being not grown caused by the effect of antibiotic

### 2-3. Principal Experiment on Antibiotic Susceptibility

### 2-3-1. Experiment Procedure

In order to determine the antibiotic resistance of the vaginosis-causing strains, i.e., Bacterioides fragilis (KCTC No. 5013) and Gardnerella vaginalis (KCTC No. 5097) as well as various lactic acid bacteria strains reproducing abundant lactic acids, i.e., Lactobacillus acidophilus (KCTC No. 3164), Bifidobacterium longum sub sp. longum (KCTC No. 3128) etc, following experiment was performed.

The growth rate of Bacterioides fragilis was determined by treating 50 microgram/ml of ampicillin to the groups treated with both Bacterioides fragilis and various Lactobacillus strains, resulting in inhibition of growth of various Lactobacillus strains.

The growth rate of Bacterioides fragilis was determined by treating 50 microgram/ml of ampicillin to the groups treated with both Bacterioides fragilis and various Bifidobacterium strains, resulting in inhibition of growth of various Bifidobacterium strains.

The growth rate of Gardnerella vaginalis was determined by treating 20 microgram/ml of ampicillin to the groups treated with both Gardnerella vaginalis and various Lactobacillus strains, resulting in inhibition of growth of various Lactobacillus strains.

The growth rate of Gardnerella vaginalis was determined by treating 20 microgram/ml of ampicillin to the groups treated with both Bacterioides fragilis and various Bifidobacterium strains, resulting in inhibition of growth of various Bifidobacterium strains.

Based on the test result from preliminary test, the test groups were divided into five groups, i.e., (a) Group I treated with the combination with refined salt and glucose (1:1, w/w), such as CL1, CF1, CB1, CS1, (b) Group II treated with the combination with melted salt and fructo-oligosaccharide (1:2, w/w), such as CL2, CF2, CB2, CS2, (c) Group III treated with the combination with unrefined salt and lactulose (1:5, w/w), such as CL3, CF3, CB3, CS3, (d) Group IV treated with the combination with refined salt and fructose (2:1, w/w), such as CL4, CF4, CB4, CS4, and (e) Group VI treated with the combination with melted salt and lactitol (5:1, w/w) such as CL5, CF5, CB5, CS5. The diluted test samples were inoculated into the selected media comprising 50 microgram/ml of ampicillin or 20 microgram of gentamycin again. 20 microliter of pre-incubated media in liquid media was diluted to 1,000 microliter of Casmans medium supplemented with 5% FBS (Fetal bovine Serum) (12 g of Tryptone, 5 g of meat peptone, 5 g of sodium chloride, 3 g of yeast extract, 3 g of beef extract, 1 g of starch casein, 0.5 g of D-glucose, 0.05 g of nicotinamide, 0.005 g of p-aminobenzoic acid, 1 L of distilled water, pH 7.3, 5% FBS) and 20 microliter of media was collected to inoculated into 3 ml of Casmans medium supplemented with 5% FBS comprising selected antibiotics again. Both of agar containing solid medium and liquid medium were performed to shaking incubation at 37° C. for 24 hrs at the speed of 150 rpm using by GasPak™EZ Pouch system (Anaerobic Gas Generation Pouch System with indicator, BD, Cat. No. 26083, USA).

The growth rate of the bacteria cultured in liquid media was determined by using spectrophotometer (Spectronic genesis 2, Thermo, USA, O. D. value at wavelength of 600 nm) and that in solid media was determined by counting the number of colonies.

### 2-3-2. Test Result

As can be seen in Table 10 showing the test result of Bacterioides fragilis in selected medium (Casmans medium: MRS medium (v/v), containing 50 microgram/ml of ampicillin), it has been confirmed that the growth of Bacterioides fragilis in the Groups I, III and V cultured with various Lactobacillus strains was inhibited and the test result on O.D (optical density) value and the counting number of colonies, was shown in Table 10.

TABLE 10
Test result on the growth of
Bacterioides fragilis cultured with various Lactobacillus strains

| B.F* | | O.D₆₀₀** | Colony No. |
|---|---|---|---|
| Group | Combination*** | 0.815 | 2862 |
| Group I | CL1 | 0.007 | 0 |
| Group II | CL2 | 0.680 | 520 |
| Group III | CL3 | 0.011 | 0 |
| Group IV | CL4 | 0.627 | 512 |
| Group V | CL5 | 0.007 | 0 |

*B.F: Bacterioides fragilis,
**O.D₆₀₀: O.D. value at wavelength of 600 nm
***combinations listed in Table 2

As can be seen in Table 11 showing the test result of Bacterioides fragilis in selected medium (Casmans medium: MRS medium (v/v), containing 50 microgram/ml of ampicillin), it has been confirmed that the growth of Bacterioides fragilis in the Groups I, II, III, IV and V cultured with Bifidobacterium strains was inhibited and the test result on O.D (optical density) value and the counting number of colonies, was shown in Table 11.

TABLE 11
Test result on the growth of
Bacterioides fragilis cultured with Bifidobacterium strains

| B.F* | | O.D₆₀₀** | Colony No. |
|---|---|---|---|
| Group | Combination*** | 0.858 | 3125 |
| Group I | CF1 | 0.050 | 0 |
| Group II | CF2 | 0.039 | 0 |
| Group III | CF3 | 0.046 | 0 |
| Group IV | CF4 | 0.045 | 0 |
| Group V | CF5 | 0.037 | 0 |

*B.F: Bacterioides fragilis,
**O.D₆₀₀: O.D. value at wavelength of 600 nm
***combinations listed in Table 2

As can be seen in Table 12 showing the test result of Bacterioides fragilis in selected medium (Casmans medium: MRS medium (v/v), containing 50 microgram/ml of ampicillin), it has been confirmed that the growth of Bacterioides fragilis in the Groups I, II, III, IV and V cultured with Bacillus cereus strain was inhibited and the test result on O.D (optical density) value and the counting number of colonies, was shown in Table 12.

TABLE 12
Test result on the growth of Bacterioides fragilis
cultured with Bacillus cereus strain

| B.F* | | O.D600** | Colony No. |
|---|---|---|---|
| Group | Combination*** | 0.841 | 3004 |
| Group I | CB1 | 0.035 | 0 |
| Group II | CB2 | 0.028 | 0 |
| Group III | CB3 | 0.041 | 0 |
| Group IV | CB4 | 0.038 | 0 |
| Group V | CB5 | 0.031 | 0 |

*B.F: Bacterioides fragilis,
O.D600**: O.D. value at wavelength of 600 nm
***combinations listed in Table 2

As can be seen in Table 13 showing the test result of Bacterioides fragilis in selected medium (Casmans medium: MRS medium (v/v), containing 50 microgram/ml of ampicillin), it has been confirmed that the growth of Bacterioides fragilis in the Groups I, II, III, IV and V cultured with Streptococcus sp strain was inhibited and the test result on O.D (optical density) value and the counting number of colonies, was shown in Table 13.

TABLE 13
Test result on the growth of
Bacterioides fragilis cultured with Streptococcus sp strain

| B.F* | | O.D₆₀₀** | Colony No. |
|---|---|---|---|
| Group | Combination*** | 0.818 | 3724 |
| Group I | CS1 | 0.031 | 0 |
| Group II | CS2 | 0.038 | 0 |
| Group III | CS3 | 0.037 | 0 |
| Group IV | CS4 | 0.027 | 0 |
| Group V | CS5 | 0.016 | 0 |

*B.F: Bacterioides fragilis,
**O.D₆₀₀: O.D. value at wavelength of 600 nm
***combinations listed in Table 2

As can be seen in Table 14 showing the test result of Gardnerella vaginalis in selected medium (Casmans medium: MRS medium (v/v), containing 20 microgram/ml of gentamycin), it has been confirmed that the growth of Gardnerella vaginalis in the Group III cultured with various Lactobacillus strain was inhibited and the test result on O.D (optical density) value and the counting number of colonies, was shown in Table 14.

TABLE 14
Test result on the growth of Gardnerella vaginalis cultured
with various Lactobacillus strains

| GV* | | O.D₆₀₀** | Colony No. |
|---|---|---|---|
| Group | Combination*** | 0.533 | 3851 |
| Group I | CL1 | 0.044 | 42 |
| Group II | CL2 | 0.031 | 33 |
| Group III | CL3 | 0.030 | 35 |
| Group IV | CL4 | 0.063 | 59 |
| Group V | CL5 | 0.061 | 55 |

*GV: Gardnerella vaginalis,
O.D₆₀₀**O.D. value at wavelength of 600 nm
***combinations listed in Table 2

As can be seen in Table 15 showing the test result of Gardnerella vaginalis in selected medium (Casmans medium: MRS medium (v/v), containing 50 microgram/ml of ampicillin), it has been confirmed that the growth of Gardnerella vaginalis in the Groups I, II, III, IV and V cultured with various Bifidobacterium strains was inhibited and the test result on O.D (optical density) value and the counting number of colonies, was shown in Table 15.

TABLE 15
Test result on the growth of
Gardnerella vaginalis cultured with Bifidobacterium strains

| *G.V: | | O.D₆₀₀** | Colony No. |
|---|---|---|---|
| Group | Combination*** | 0.831 | 3125 |
| Group I | CF1 | 0.036 | 0 |
| Group II | CF2 | 0.031 | 0 |
| Group III | CF3 | 0.035 | 0 |
| Group IV | CF4 | 0.028 | 0 |
| Group V | CF5 | 0.025 | 0 |

*GV: Gardnerella vaginalis,
**O.D₆₀₀: O.D. value at wavelength of 600 nm
***combinations listed in Table 2

As can be seen in Table 16 showing the test result of Gardnerella vaginalis in selected medium (Casmans medium: MRS medium (v/v), containing 50 microgram/ml of ampicillin), it has been confirmed that the growth of Gardnerella vaginalis in the Groups I, II, III, IV and V cultured with Bacillus cereus strain was inhibited and the test result on O.D (optical density) value and the counting number of colonies, was shown in Table 16.

TABLE 16
Test result on the growth of
Gardnerella vaginalis cultured with Bacillus cereus strain

| G.V* | | O.D₆₀₀** | Colony No. |
|---|---|---|---|
| Group | Combination*** | 0.856 | 3447 |
| Group I | CB1 | 0.035 | 0 |
| Group II | CB2 | 0.021 | 0 |
| Group III | CB3 | 0.026 | 0 |
| Group IV | CB4 | 0.015 | 0 |
| Group V | CB5 | 0.026 | 0 |

*GV: Gardnerella vaginalis,
**O.D₆₀₀: O.D. value at wavelength of 600 nm
***combinations listed in Table 2

As can be seen in Table 17 showing the test result of Gardnerella vaginalis in selected medium (Casmans medium: MRS medium (v/v), containing 50 microgram/ml of ampicillin), it has been confirmed that the growth of Gardnerella vaginalis in the Groups I, II, III, IV and V cultured with Streptococcus sp strain was inhibited and the test result on O.D (optical density) value and the counting number of colonies, was shown in Table 17.

TABLE 17
Test result on the growth of
Gardnerella vaginalis cultured with Streptococcus sp strain

| GV* | | O.D₆₀₀** | Colony No. |
|---|---|---|---|
| Group | Combination*** | 0.832 | 3486 |
| Group I | CS1 | 0.025 | 0 |
| Group II | CS2 | 0.027 | 0 |
| Group III | CS3 | 0.031 | 0 |
| Group IV | CS4 | 0.019 | 0 |
| Group V | CS5 | 0.010 | 0 |

*GV: Gardnerella vaginalis,
**O.D₆₀₀: O.D. value at wavelength of 600 nm
***combinations listed in Table 2

Accordingly, it has been confirmed that the inventive combination of the present invention showed more potent inhibiting effect on vaginosis-causing bacteria comparing with the sole treatment of lactic acid bacteria and the combination of salt and sugar through the above experiments.

### Experimental Example 3. Brief Clinical Test (1)

1,200 mg of the vaginal tablet composition (SGL2) prepared in Example 2 was administrated externally once a day for 5 days to 100 volunteers consisting of 35 patients suffering from vaginosis, and 65 normal women ranging from 20 to 50 years who live in Korea to conduct a questionnaire survey and the difference of various contents, (a) preventive effect from unpleasant scent, (b) the level of freshness, (c) and the alleviating activity of skin psoriasis was surveyed.

The investigated result was classified into four groups, (1) very satisfied, (2) satisfied, (3) normal and (4) unsatisfied and the surveyed result shown in Table 18.

TABLE 18
Result of survey
Surveyed content

| Content | very satisfied | satisfied | normal | unsatisfied | sum |
|---|---|---|---|---|---|
| (a) | 79 | 16 | 5 | 0 | 100 |
| (b) | 74 | 16 | 8 | 2 | 100 |
| (c) | 71 | 19 | 10 | 0 | 100 |

At the result, as can be seen in Table 18, as to the (a) preventive effect from unpleasant scent, 94% volunteers after the treatment with inventive composition was satisfied and in particular, 79% volunteers was very satisfied.

As to the (b) the level of freshness, 86% volunteers after the treatment with inventive composition were satisfied and in particular, 72% volunteers were very satisfied. As to the alleviating activity of skin psoriasis, 85% volunteers after the treatment with inventive composition were satisfied and in particular, 67% volunteers were very satisfied.

Accordingly, it has been proved that the inventive vaginal tablet composition has potent favorable effect, for example, (a) preventive effect from unpleasant scent, (b) the level of freshness, (c) and the alleviating activity of skin psoriasis etc and it can be useful as a vaginal tablet composition for treating or preventing the patients from vaginal vaginosis.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### Experimental Example 4. Brief Clinical Test (2)

200 ml of the vaginal cleansing composition (SGL4) prepared in Example 3 was administrated externally once a day for 5 days to 100 volunteers consisting of 42 patients suffering from vaginosis, and 58 normal women ranging from 20 to 50 years who live in Korea and the difference of vaginal pH between the pH of (A) before and (B) after the treatment with inventive composition was determined using by pH meter (MP-103, www.yuyuinst.co.kr).

At the result, as can be seen in Table 19, the vaginal pH of 85% test group before the treatment with inventive composition had reached to more than 5.5 however that of 90% test group after the treatment with inventive composition reached to normal pH range.

TABLE 19
pH difference

| | pH | | | | | | | |
|---|---|---|---|---|---|---|---|---|
| | <3.5 | 4 | 4.5 | 5 | 5.5 | 6 | >6.5 | Sum |
| A | 0 | 1 | 7 | 8 | 17 | 49 | 19 | 100 |
| B | 1 | 22 | 45 | 23 | 8 | 1 | 0 | 100 |

Accordingly, it has been proved that the inventive cleansing composition can be SGL4 can be useful in decreasing the vaginal pH of the patients suffering with vaginal akalisation.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### Experimental Example 5. Acute Toxicity Test of Oral Administration in Rat

The acute toxicity test was performed by administrating inventive combinations (SGL 2 and SGL4) to 6-weeks aged SPF Sprague-Dawley rats.

250 mg/kg, 500 mg/kg, 1000 mg/kg, 5000 mg/kg of inventive combination was orally administrated to each group consisting of 2 rats and the symptoms of rats were observed for 14 days. After administrating the inventive combinations, all the clinical changes i.e., mortality, clinical signs, body weight changes was observed and blood test such as haematological test and hematological biochemistry test was performed. The abnormal changes of abdominal organ and thoracic organ were observed after autopsy.

There did not show any changes in mortality, clinical signs, body weight changes and gross findings in any group or either gender. Furthermore, there showed any toxicity in test group treated with 5000 mg/kg of inventive combinations.

Accordingly, it has been confirmed that the inventive combinations prepared in the present invention was potent and safe substance showing LD₅₀ (more than 5000 mg/kg) in oral administration.

### MODE FOR INVENTION

Hereinafter, the formulating methods and kinds of excipients will be described, but the present invention is not limited to them. The representative preparation examples were described as follows.

### Preparation of Injection

CB5 combination (100 mg)
Sodium metabisulfite (3.0 mg)
Methyl paraben (0.8 mg)
Propyl paraben (0.1 mg)
Distilled water for injection optimum amount

Injection preparation was prepared by dissolving active component, controlling pH to about 7.5 and then filling all the components in 2 ml ample and sterilizing by conventional injection preparation method.

### Preparation of Powder

CL1 combination (500 mg)
Corn Starch (100 mg)
Lactose (100 mg)
Talc (10 mg)

Powder preparation was prepared by mixing above components and filling sealed package.

### Preparation of Tablet

CL3 combination (200 mg)
Corn Starch (100 mg)
Lactose (100 mg)
Magnesium stearate optimum amount

Tablet preparation was prepared by mixing above components and entabletting.

### Preparation of Capsule

CB3 combination (100 mg)
Lactose (50 mg)
Corn starch (50 mg)
Talc (2 mg)
Magnesium stearate optimum amount

Tablet preparation was prepared by mixing above components and filling gelatin capsule by conventional gelatin preparation method.

### Preparation of Liquid

CS3 combination (1000 mg)
Sugar (20 g)
Polysaccharide (20 g)
Lemon flavor (20 g)

Liquid preparation was prepared by dissolving active component, and then filling all the components in 1000 ml ample and sterilizing by conventional liquid preparation method.

### Preparation of Health Food

CS5 combination (1,000 mg)
Vitamin mixture (optimum amount)
Vitamin A acetate (70 g)
Vitamin E (1.0 mg)
Vitamin B₁₀ (13 mg)
Vitamin B₂ (0.15 mg)
Vitamin B6 (0.5 mg)
Vitamin B1 (20.2 mg)
Vitamin C (10 mg)
Biotin (10 mg)
Amide nicotinic acid (1.7 mg)
Folic acid (50 mg)
Calcium pantothenic acid (0.5 mg)
Mineral mixture (optimum amount)
Ferrous sulfate (1.75 mg)
Zinc oxide (0.82 mg)
Magnesium carbonate (25.3 mg)
Monopotassium phosphate (15 mg)
Dicalcium phosphate (55 mg)
Potassium citrate (90 mg)
Calcium carbonate (100 mg)
Magnesium chloride (24.8 mg)

The above mentioned vitamin and mineral mixture may be varied in many ways. Such variations are not to be regarded as a departure from the spirit and scope of the present invention.

### Preparation of Health Beverage

CL4 combination (1000 mg)
Citric acid (1000 mg)
Oligosaccharide (100 g)
Apricot concentration (2 g)
Taurine (1 g)
Distilled water (900 ml)

Health beverage preparation was prepared by dissolving active component, mixing, stirred at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the present invention provides a composition comprising an inventive combination of salt, sugar and lactic acid bacteria as an active ingredient to treat or prevent vaginosis. The inventive composition showed potent antibacterial activity through various experiments, for example, (1) the indirect inhibitory activity test from the growth of vaginosis causing bacteria by determining the change of pH and lactic acid level (Experimental example 1); (2) the direct inhibitory activity test from the growth of vaginosis causing bacteria by determining the susceptibility of test sample (Experimental example 2); (3) brief clinical tests, and finally confirmed that the combination showed potent antibacterial activity in the test. Accordingly, the inventive combination may be useful to alleviate, treat or prevent vaginosis in the form of a pharmaceutical composition, health functional food, food additive, topical composition, and detergent composition.

1.-17. (canceled)

18. A composition comprising salt, sugar and lactic acid bacteria.

19. The composition of claim 18, wherein the salt is selected from the group consisting of a refined salt, a processed salt and an un-refined salt.

20. The composition of claim 18, wherein the salt is sodium chloride.

21. The composition of claim 18, wherein the sugar is selected from the group consisting of a mono-saccharide, a disaccharide, an oligosaccharide and a polysaccharide.

22. The composition of-claim 18, wherein the sugar is selected from the group consisting of xylose, arabinose, glucose, mannose, fructose, galactose, lactulose, lactitol, sucrose, lactose, maltose, trehalose, fructo-oligosaccharide, raffinose, stachyose, maltodextrin, amylose, cellulose and pectin.

23. The composition of claim 18, wherein the salt, sugar and lactic acid bacteria are in a mixed ratio of 1:100-0.01:100-0.01 (w/w).

24. The composition of claim 18, wherein the lactic acid bacteria is selected from the group consisting of Lactobacillus spp., Bifidobacterium spp., Bacillus spp., Streptococcus spp., Enterococcus spp., Saccharomyces spp., and Leuconostoc spp.

25. The composition of claim 24, wherein the Lactobacillus spp. bacteria is selected from the group consisting of lactobacillus plantarum, lactobacillus pentosus, lactobacillus casei, lactobacillus casei ssp. paracasei, lactobacillus rhamnosus, lactobacillus acidophilus, lactobacillus delbrueckii, lactobacillus delbrueckii ssp. bulgaricus, lactobacillus delbrueckii ssp. delbrueckii, lactobacillus fermentum, lactobacillus gasseri, lactobacillus reuteri, lactobacillus brevis, lactobacillus cellobiosus, lactobacillus crispatusi, lactobacillus GG, lactobacillus johnsonii, lactobacillus lactis, and lactobacillus salivarius.

26. The composition of claim 25, wherein the Lactobacillus spp. bacteria is selected from the group consisting of lactobacillus plantarum, lactobacillus pentosus, lactobacillus casei, lactobacillus casei ssp. paracasei, lactobacillus rhamnosus, and lactobacillus acidophilus.

27. The composition of claim 24, wherein the Bifidobacterium spp. bacteria is selected from the group consisting of Bifidobacterium longum, Bifidobacterium bifidum, Bifidobacterium bereve, Bifidobacterium animalis ssp. lactis, Bifidobacterium adoescentis, Bifidobacterium pseuocatenulatum, Bifidobacterium catenulatum, Bifidobacterium infantis, and Bifidobacterium thermophilum.

28. The composition of claim 18, wherein the composition is a pharmaceutical composition.

29. The composition of claim 28, wherein the pharmaceutical composition further comprises a pharmaceutically acceptable carrier.

30. The composition of claim 18, wherein the composition is a health functional food.

31. The composition of claim 30, wherein the health functional food is selected from the group consisting of a food, a health beverage and a dietary supplement.

32. The composition of claim 30, wherein the health functional food further comprises a sitologically acceptable additive.

33. The composition of claim 18, wherein the composition is incorporated into a form selected from the group consisting of topical composition, detergent composition, liquid solution and insert preparation.

34. The composition of claim 33, wherein the topical composition is selected from the group consisting of a aqueous solution, non-aqueous solvent, suspension, emulsion, lyophilized preparation, suppository preparation, cleansing liquid, gel, jelly, foam, cream, ointment, lotion, balm, patch, paste, spray solution and aerosol.

35. The composition of claim 33, wherein the insert preparation is selected from the group consisting of vaginal tablet, vaginal cream, vaginal ointment, dressing solution, spraying preparation, vaginal capsule, vaginal film, vaginal sponge, spreading cream, tampon, sanitary pad, diaper and panties.

36. A method of treatment comprising administering to a mammal in need of treatment for vaginosis an effective amount of the composition of claim 18, together with a pharmaceutically acceptable carrier thereof.

37. The method of claim 36, wherein the vaginosis is selected from the group consisting of bacterial vaginosis, fungal vaginosis and Tricomonas vaginitis.
